SEQUENCE LISTING (5 sequences)

SEQ ID NO: 1 - DNA, 2542 bp, Penicillium chrysogenum
agctgaagaatagatgaatttctatgttgcgaataatagtagtttccaatactacaaatttggagagtcattaagtactaaaatgtttggatatccccacctagcgtggggattgggtcagtgctatttgacccgatcgcgtcgcaaatgcaacccgatcggatcggagtccggtcttcgcggtcgtattcagacatctagcggggccatcaatagcgcgtggtaacgcatgacactgcgtggcgtcgcatgtctgattcagtggttcgcttcagttcttcaacgttgcggtcaatgagattgtggtaacgcagtcagcatacagatcctgtgaagctaaaccagcagagccagtgggaccagtgggaccagtggataccttccactccccagggtctgcattttccggggaaaccgacgtaagccaaggaactcaaaattggtatgtactatctatcctctacccgtcaatacatactatgtgttattggatttccctctatcaaatcgaatagactgcatagattgcagtcctttcctttcgtcagattatccagattatccagatcagtcccgaaagtgaaactgaatccaatcttcgatataaatagctccttgttcctccaaaaaaacaagagtcatcatctcagttagctgtttccacacttcctctttcactcaaaagcccgctttttcaggtcaattttagtggactaacttcacgatgctatcttcattgatgtatttggcacgtgagtgctccatcttgcgcgctatctccttatgtgccatcctcgccgtggcaactcctgcggcgagcgcaagcgtcacagcaaagcacacgctggccacaaatggaaaggggctgtttccacagtaccgaattgttgccttggcaagtctgggaaatggtgttctcctggcctcatatgatgggcgcccagatggaggagattcgccatccccaaattcaattctacaacgacgcagtacggacggtgggaagacttggggcaacccaacatatattgcgcgaggtcaaccagcgtcgtcgacactccaacagtacggctttagtgacccaagctacgtggtcgactccggtactggaaaggtctttaatttccatgtcttctcgaagaaccagggatttctcaatagcgaaattggaaatgacgacaccgacttaaacatagtcagcgctgaagtctccgtttcgactgatggaggacttagctggaccactgatccagaccatgaatcctctttgcctcccgttgcatctgccgacgttggtgcaccgccactcattacgaaagcaatcaagcccgtgggtagtacttccaacggggtagccaacgtcggtggaattactgggatgtttgcctcatccggagaggggattcaactcaaatacggcaaacacgccgggcgcctggttcaacaattccttgggaaagtcatccaatcagacggttcaaaggtctcgcaagcctacagtgtctacagtgacgacggtggcgctatatggaagatggggaaagtcattggcactgggatggatgagaacaaagttgtggagctatcaaacggcaacttgatgcttaactcacgcccgagcgatggtagcggatatcgaaaggtggcaacctcaaccgatggtggtgaaacttggtcaacaccagcaagcgaaacccaactccctgacccaggaaataacggagcaattaccaggatgtatcctgatgcggcacatggttcggccaatgccaagatcctcctgtttaccaatgcaaacagtaaaacaagccgaagcaatggtacaatccgctattcctgtgacgacggaaagacctggtcttctggcgcagtgttccagtctggctcgatgtcctattccactgtcaccgcactcggcgatgacaggttcggaatattttacgagggggatagcaacgacctcgtctacattgaagtttccaaggattttattggggtttcctgctgataaaactcccattggcagtgtgctctacttgggaacttggtttttacattgtacctaggtctatgtgtacatgtatgtactacagcgtcatctcaaatattcttttgggaaaggacctgacaatggcggcagcatgatggattttgtggctcggccatttattgacgatggcacacgatagctatcgaactcactggtagctattgcgaatgttcagtacaggtggcgttggttcctctagtcacgcctggatgaaatcagaccgttatagtgacaccttcctatgacactatattctgtattgtgaacccaatatttccatggtagtagtttcaggtgacagcaagggcaaaaattcttattgctcaaaagtaacattgcatgtagagatcccatagtcaaggtggcgtttgagagatttataggtggtaaaatcatgctatttttaggcttaggaacaatgctcgcaagacgaacgaacgatgtagtaggttggattagagcccagtaagaatggaattcctatcacaagca

SEQ ID NO: 2 - DNA, 1224 bp, Penicillium chrysogenum
  CDS (1)..(1224)
atgctatcttcattgatgtatttggcacgtgagtgctccatcttgcgcgctatctccttatgtgccatcctcgccgtggcaactcctgcggcgagcgcaagcgtcacagcaaagcacacgctggccacaaatggaaaggggctgtttccacagtaccgaattgttgccttggcaagtctgggaaatggtgttctcctggcctcatatgatgggcgcccagatggaggagattcgccatccccaaattcaattctacaacgacgcagtacggacggtgggaagacttggggcaacccaacatatattgcgcgaggtcaaccagcgtcgtcgacactccaacagtacggctttagtgacccaagctacgtggtcgactccggtactggaaaggtctttaatttccatgtcttctcgaagaaccagggatttctcaatagcgaaattggaaatgacgacaccgacttaaacatagtcagcgctgaagtctccgtttcgactgatggaggacttagctggaccactgatccagaccatgaatcctctttgcctcccgttgcatctgccgacgttgccaacgtcggtggaattactgggatgtttgcctcatccggagaggggattcaactcaaatacggcaaacacgccgggcgcctggttcaacaattccttgggaaagtcatccaatcagacggttcaaaggtctcgcaagcctacagtgtctacagtgacgacggtggcgctatatggaagatggggaaagtcattggcactgggatggatgagaacaaagttgtggagctatcaaacggcaacttgatgcttaactcacgcccgagcgatggtagcggatatcgaaaggtggcaacctcaaccgatggtggtgaaacttggtcaacaccagcaagcgaaacccaactccctgacccaggaaataacggagcaattaccaggatgtatcctgatgcggcacatggttcggccaatgccaagatcctcctgtttaccaatgcaaacagtaaaacaagccgaagcaatggtacaatccgctattcctgtgacgacggaaagacctggtcttctggcgcagtgttccagtctggctcgatgtcctattccactgtcaccgcactcggcgatgacaggttcggaatattttacgagggggatagcaacgacctcgtctacattgaagtttccaaggattttattggggtttcctgctga

SEQ ID NO: 3 - protein, 407 aa, Penicillium chrysogenum
MLSSLMYLARECSILRAISLCAILAVATPAASASVTAKHTLATNGKGLFPQYRIVALASLGNGVLLASYDGRPDGGDSPSPNSILQRRSTDGGKTWGNPTYIARGQPASSTLQQYGFSDPSYVVDSGTGKVFNFHVFSKNQGFLNSEIGNDDTDLNIVSAEVSVSTDGGLSWTTDPDHESSLPPVASADVANVGGITGMFASSGEGIQLKYGKHAGRLVQQFLGKVIQSDGSKVSQAYSVYSDDGGAIWKMGKVIGTGMDENKVVELSNGNLMLNSRPSDGSGYRKVATSTDGGETWSTPASETQLPDPGNNGAITRMYPDAAHGSANAKILLFTNANSKTSRSNGTIRYSCDDGKTWSSGAVFQSGSMSYSTVTALGDDRFGIFYEGDSNDLVYIEVSKDFIGVSC

SEQ ID NO: 4 - DNA, 46 bp, Artificial Sequence
  synthetic primer for ZJW coding region starting at the ATG start codon, preceded by a 23 nucleotides sequence including a PacI site
cccttaattaactcataggcatcatgctatcttcattgatgtattt

SEQ ID NO: 5 - DNA, 34 bp, Artificial Sequence
  synthetic primer complementary to the reverse strand of the region downstream of the ZJW coding region preceded by an AscI site
ttaggcgcgccgtacatacatgtacacatagacc

(19) United States
(12) Patent Application Publication
(10) Pub. No.: US 2012/0294980 A1
(43) Pub. Date: Nov. 22, 2012

(54) NOVEL PREBIOTICS

(75) Inventors: Albertus Alard VAN DIJK, Vlaardingen (NL); Yulia M. Efimova, Delft (NL); Margot Elisabeth Francoise SCHOONEVELD-BERGMANS, Delft (NL); Natalja Alekseevna Cyplenkova, Vlaardingen (NL)

(73) Assignee: DSM IP ASSETS B.V., HERLEEN (NL)

(21) Appl. No.: 13/567,870

(22) Filed: Aug. 6, 2012

Related U.S. Application Data
(63) Continuation of application No. 12/666,975, filed on Dec. 28, 2009, now abandoned.

(30) Foreign Application Priority Data
Jun. 25, 2007 (EP) 07110983.9
Mar. 25, 2008 (EP) 08102295.6

(51) Int. Cl. C12P 19/26; A23L 1/30

(52) U.S. Cl. 426/48; 435/84; 426/71; 426/61

(57) ABSTRACT

The present invention relates to a composition which comprises:
an oligosaccharide which is free of sialyloligosaccharide, and
free sialic acid.

### CROSS REFERENCE RELATED APPLICATION

This is a continuation application of U.S. Ser. No. 12/666,975 filed Dec. 28, 2009 which is a U.S. National Phase of PCT/EP2008/057948 filed Jun. 23, 2008 which claims priority to EP Application No. 08102295.6 filed Mar. 5, 2008 and EP Application No. 07110983.9 filed Jun. 25, 2007, the entire contents of which each are incorporated by reference in their entireties.

### FIELD OF THE INVENTION

This invention relates to a method for the preparation of novel prebiotic compositions.

### BACKGROUND OF THE INVENTION

Prebiotics

A prebiotic is a non-digestible food ingredient that beneficially affects the host by selectively stimulating the growth and/or activity of one of a limited number of bacteria in the colon, and thus improves health. In general, mammalians, preferably humans, can take advantage of prebiotics. Prebiotics mostly are short chain carbohydrates that alter the composition, or metabolism, of the gut microflora in a beneficial manner. The short chain carbohydrates are also referred to as oligosaccharides, and usually contain between 3 and 10 sugar moieties or simple sugars. When oligosaccharides are consumed, the undigested portion serves as food for the intestinal microflora. Depending on the type of oligosaccharide, different bacterial groups are stimulated or suppressed.

Oligosaccharides prepared for use in the food industry are not single components, but are mixtures containing oligosaccharides with different degrees of oligomerization, sometimes including the parent disaccharide and the monomeric sugars (Prapulla et al. (2000) Adv Appl microbial 47, 299-343). Various types of oligosaccharides are found as natural components in many common foods, including fruits, vegetables, milk, and honey. Examples of oligosaccharides are galacto-oligosaccharides, lactulose, lactosucrose, fructooligosaccharides, palatinose or isomaltose oligosaccharides, glycosyl sucrose, maltooligosaccharides, isomaltooligosaccharides, cyclodextrins, gentiooligosaccharides, soybean oligosaccharides and xylooligosaccharides (Prapulla et al. (2000) Adv Appl microbial 47, 299-343).

Candidate oligosaccharides, of which the prebiotic potential has been investigated limitedly, are derived from germinated barley, dextrans, pectins, polygalacturonan, rhamnogalacturonan, mannan, hemicellulose, arabinogalactan, arabinan, arabinoxylan, resistant starch, melibiose, chitosan, agarose, alginate (Van Loo, 2005, Food Science and Technology Bulletin: Functional Foods 2: 83-100; Van Laere et al., 2000, J Agric Food Chem 48, 1644-1652; Lee et al. 2002, Anaerobe 8, 319-324; Hu et al, 2006, Anaerobe 12, 260-266; Wang et al, 2006, Nutrition Research 26, 597-603). All of these oligosaccharides are produced using enzymatic processes involving either the hydrolysis of polysaccharides or the synthesis starting from smaller carbohydrates using transglycosylation reactions. In some cases hydrothermal treatment or autohydrolysis is applied to depolymerise lignocellulosic materials, such as xylans (Vazquez et al, 2006, Industrial Crops and Products, 24, 152-159).

Three prebiotics, oligofructose (inulin), transgalacto-oligosaccharides and lactulose clearly alter the balance of the large bowel microbiota by increasing Bifidobacteria and Lactobacillus numbers (A. Singh et al: The future aspects of prebiotics on human health, A review. www.pharmainfo.net; van Loon Food Sci Technol Bull: Functional Foods 2, 83-100). Inulin, fructo-oligosaccharides (FOS), galacto-oligosaccharides and lactulose, when taken in the diet in relatively small amounts (5-20 g/day) have been clearly shown in human studies to stimulate growth of health promoting species belonging to the genera Bifidobacterium and Lactobacillus. These are ordinarily not the most numerous organisms in the gut except in the breastfed baby (A. Singh et al: The future aspects of prebiotics on human health, A review. www.pharmainfo.net and references sited therein). This selective growth stimulation of Bifidobacteria and Lactobacilli by prebiotics is supposed to be at the expense of the growth of other bacteria in the gut, such as Bacteroides, Clostridia, eubacteria, enterobacteria, enterococci etc., although so far there is no firm quantification of these effects.

The micro-biota of the human intestinal tract plays an important role in health, in particular by mediating many of the effects of diet upon gut health. The human large intestine is colonized by a dense and complex community composed of largely anaerobic bacteria. The activities of these organisms have a major impact upon the nutrition and health of the host via the supply of nutrients, conversion of metabolites and interactions with host cells. The energy sources that support the microbial community of the large intestine are dietary components that resist degradation in the upper intestinal tract, together with endogenous products like mucin. Anaerobic metabolism by the microbial community in the colon produces short-chain fatty acids together with carbondioxide, hydrogen and methane. (Flint et al, Env Microbiol (2007) 9, 1101-1111). These have significant effects on the gut environment and on the host as energy sources, regulators of gene expression, cell differentiation and anti-inflammatory agents. There is increasing evidence that bacterial populations in the large intestine respond to changes in diet, in particular to the type and quantity of dietary carbohydrate. (Flint et al, Env Microbiol (2007) 9, 1101-1111). Changes in the type and quantity of non-digestible carbohydrates in the human diet influence both the metabolic products formed in the lower regions of the gastrointestinal tract and bacterial populations detected in the faeces. Non-digestible carbohydrates such as inulin, fructo-oligosaccharides and galacto-oligosaccharides are now widely used as prebiotics in order to manipulate the composition of the gut microbiota. A range of other naturally occurring oligosaccharides, and also synthetic products, have selective effects in vitro (Manderson et al, 2005, Appl Environ Microbiol 71, 8383-8389). Prebiotic effects are likely to be influenced by many features of the substrate, including solubility, the distribution of chain lengths, branching and substituents (Rossi et al, 2005, Appl Environ Microbiol 71, 6150-6158). Tests of the ability of isolated bacteria to utilize purified carbohydrates in vitro can provide a preliminary indication of substrate preferences in mixed eco-systems like the gut. It is expected that responses to prebiotics will depend on the dietary context and the gut environment and will be influenced by variations in the species composition and the resident gut microbiota between individuals.

Prebiotic oligosaccharides have been shown to confer a variety of health promoting effects. Although not all of them have been fully demonstrated, the following beneficial effects have been postulated (Swennen et al, Crit. Rev. Food Sci Nutr. 2006, 46, 459-471): alleviation of constipation, improvement of mineral absorption, regulation of lipid metabolism, decrease in risk of colon cancer, beneficial in treatment of hepatic encephalopathy, positive effect on glycemia/insulinemia and modulation of the immune system of the intestine. Prebiotic oligosaccharides have never been demonstrated to have a positive effect on learning ability, memory formation and brain development.

Production of Oligosaccharides

The production of oligosaccharides has been described in literature. Since the chemical synthesis of oligomeric sugars is notoriously difficult, enzymes are usually used to prepare oligosaccharides. Exceptions are situations in which isomerization can be used, e.g. in the case of lactulose production. Enzymes can be used either in the free form without restriction of movement in the reaction mixture. Alternatively, enzymes can be immobilized on a suitable carrier, restricting their movement in the reaction system. Immobilization can be obtained by covalent coupling of the enzyme to a carrier substrate or by physical entrapment of the enzyme in e.g. a gel matrix. Methods to immobilize enzymes are known to the expert in the field; reviews have appeared on this topic. (see e.g. Mateo et al 2007, Enz. Micr. Technol. 40, 1451-1463). Enzymes may also be cross-linked to form large aggregates that can easily be separated from the reaction mature by filtration (see for review e.g. Margolin et al, 2001, Angew. Chem. Int. Ed. 40, 2204-2222).

Galacto-oligosaccharides are produced commercially from lactose using the galactosyltransferase activity of β-galactosidase, which dominates lactose hydrolysis at high lactose concentrations. This process has been described in detail, and excellent reviews have appeared on this topic (see e.g. Mahoney, 1998, Food Chem. 63, 147-154; Zarate et al 1990, J Food protection 53, 262-268). Various β-galactosidases have been described that can be used for the oligomerization process, and in several occasions the reaction products have also been described (see e.g. Burvall et al 1979, Food Chem 4, 243-249; Asp et al 1980, Food Chem 5, 147-153).

Lactulose is produced by an alkaline isomerization process that converts the glucose moiety in lactose to a fructose residue.

Lactosucrose is manufactured from a mixture of lactose and sucrose using the transfructosylation activity of the enzyme β-fructofuranosidase.

Fructose oligosaccharides are manufactured by two different processes. One is from the disaccharide sucrose using the transfructosylation activity of the enzyme β-fructofuranosidase, the other one is via the controlled enzymatic hydrolysis of inulin with inulinase.

Palatinose or isomaltulose oligosaccharides are produced from sucrose using immobilized isomaltose synthase.

Glycosyl sucrose is manufactured from maltose and sucrose using the enzyme cyclomaltodextrin glucanotransferase.

Maltooligosaccharides are produced from starch by the action of debranching enzymes such as pullulanase and isoamylase, combined with hydrolysis by various α-amylases.

Processes for the production of isomaltooligosaccharides, cyclodextrins, gentiooligosaccharides, soybean oligosaccharides and xylooligosaccharides have also been developed (for reference see Prapulla et al. (2000) Adv Appl Microbial 47, 299-343 and references sited therein).

Sialic acid means N-acetylneuraminic acid (Neu5Ac or NANA). Free sialic acid means sialic acid which is bound or part of another compound.

Sialyloligosaccharides

Exclusively breast-fed neonates have a microbiota containing proportionally higher numbers of Bifidobacteria, which is believed to be part of the baby's defense against pathogenic micro-organisms an which may be important primers for their immune system. This microbiota is nurtured by oligosaccharides in breast milk, which can be considered to be the original prebiotics. Of special interest is that mothers breast milk contains relatively high levels of sialyloligosaccharides. The concentration of such oligosaccharides is substantially lower in cow's milk, which is often used to prepare infant nutrition. Several patents describe how the levels of such sialyloligosaccharides in cows milk can be increased (e.g. U.S. Pat. No. 6,706,497; U.S. Pat. No. 5,374,541; U.S. Pat. No. 5,409,817). Sialyllactose has been described to neutralize enterotoxins of various pathogenic microbes including Escherichia coli, Vibrio cholerae, and Salmonella (U.S. Pat. No. 5,330,975). Other beneficial effects on the gut population of sialic-acid containing oligosaccharides have been described, including the interference with colonization by Helicobacter pylori (see e.g. U.S. Pat. No. 5,514,660; U.S. Pat. No. 5,164,374). These sialic acid containing carbohydrates can therefore also been classified as prebiotcis since they beneficially affect the host by selectively influencing the gut microbiota. Synonyms for sialyloligosaccharides are sialic acid-rich oligosaccharides or oligosaccharide-bound sialic acid

Sialic Acid

Sialic acids comprise a family of about 40 derivatives of the nine-carbon sugar neuraminic acid. It is a strong organic acid with a pKₐ of around 2.2. The unsubstituted form, neuraminic acic, does not exist in nature. The amino group is usually acetylated to yield N-acetylneuraminc acid, the most widespread form of sialic acid, but other forms exist as well (Traving et al Cell Mol Life Sci (1998) 54, 1330-1349). Sialic acids have been found in the animal kingdom, from the echinoderms upwards to humans whereas there is no hint for their existence in lower animals of the protostomate lineage or in plants. The only known exception is the occurrence of polysialic acid in larvae of the insect Drosophila. In addition there are sialic acids in some protozoa, viruses and bacteria. Sialoglycoconjugates are present on cell surfaces as well as in intracellular membranes. In higher animals they are also important components of the serum and of mucous substances.

Sialic acids have a variety of biological functions. Due to their negative charge sialic acids are involved in binding and transport of positively charged molecules like calcium ions, as well as in attraction and repulsion phenomena between cells and molecules. Their exposed terminal position in carbohydrate chains, in addition to their size and negative charge enable them to function as a protective shield for the sub-terminal part of the molecule or the cell. They can e.g. prevent glycol-proteins from being degraded by proteases or the mucous layer of the respiratory system from bacterial infection. An interesting phenomenon is the spreading effect that is exerted on sialic acid containing molecules due to the repulsive forces acting between their negative charges. This stabilizes the correct conformation of enzyme or membrane (glyco)-proteins, and is important for the slimy character and the resulting gliding and protective function of mucous substances, such as on the surface to the eye or on mucous epithelia (Traving et al Cell Mol Life Sci (1998) 54, 1330-1349).

Sialic acids take part in a variety of recognition processes between cells and molecules. Thus, the immune system can distinguish between self and non-self structures according to their sialic acid pattern. The sugar represents an antigenic determinant, for example blood group substances, and is a necessary component of receptors for many endogenous substances such as hormones and cytokines. In addition, many pathogenic agents such as toxins (e.g. cholera toxin), viruses (e.g. influenza) bacteria (e.g. Escherichia coli, Helicobacter pylori) and protozoa (e.g. Trypanosome cruzi) also bind host cells via sialic acid-containing receptors. Another important group of sialic acid recognizing molecules belong to the lectins, which are usually oligomeric glycoproteins from plants, animals and invertebrates that bind specific sugar residues. Examples are wheat germ agglutinin, Limulus polyphemus agglutinin, Sambucus nigra agglutinin and Maackia amurensis agglutinin. These lectins seem to help the plant in its defense against sialic acid containing micro-organisms or plant-eating mammals. Mammalian counterparts of the lectins include selectins and siglecs (Traving et al Cell Mol Life Sci (1998) 54, 1330-1349) and have a variety of physiological roles. Sialic acids can also assist in masking of cells and molecules. Erythrocytes are covered by a dense layer of sialic acid molecules, which is stepwise removed during the life cycle of the blood cell. The penultimate galactose residue that represent signals for degradation than become visible and the unmasked blood cells are than bound to macrophages and phagocytosed. Several other examples of such masking strategy are known. Masking can also have a detrimental effect, as can be seen from some of the tumors that are sialylated to a much higher degree than the corresponding tissues. Consequently, the masked cells are invisible to the immune defence system, and the high sialic acid contents may also play a role in the lack of inhibition of further cell growth and in spreading. The masking effect of sialic acids also helps to hide antigenic sites on parasite cells, making them invisible for the system. This is the case for microbial species like certain E. coli strains and gonococci (Neisneria gonorrhoeae).

Sialic Acid as an Emerging Prebiotic

The importance of sialic acid based oligosaccharides derived from the glyco macro peptide (GMP) with respect to prevention of infection was shown in case of using it as emerging prebiotics (K. M. Tuohy G. C. M. Rouzaud Current Pharmaceutical Design, 2005, 11, 75-90). Additionally it was reported that sialic acid containing GMP derived from human milk was an effective growth-promoting factor for bifidobacteria and had several anti-pathogenic attributes (W. M. Bruck FEMS Microbial Ecol 2002; 41:231-7). In a recent review article (Sakaki Ken et al, Food Style 21 (2002), 6(1), 64-67) the characteristics and applications of sialic acid and sialyl-oligosaccharides derived from eggs are described. From literature data it can be concluded that sialic acid in combination with proper oligosaccharides can be used as successful prebiotics. The preparations described are always mixtures of sialic acid and sialic acid-containing oligosaccharides. Preparations containing free sialic acid and non-sialylated oligosaccharides have to our knowledge not been described.

Another very important feature of sialic acid is its effect on brain development, learning ability and memory formation in animal studies. It was reported that variations in the sialic acid content of a formula milk clearly influences early learning behavior and gene expression of enzymes involved in sialic acid metabolism (B. Wang et al Am. J. Clin Nutr, 2007, 85, 561-569). At the same time, the concentration of sialic acid in brain ganclicosides and glycoproteins was directly linked to amount of free sialic acid fed to rat pups (S. E. Carlson, S. G. House The Journal of nutrition, 1986, 881-886).

Human milk is a rich source of sialic acid and from the human studies it was found that concentration of sialic acid in the brain frontal cortex of breast fed infants was significantly higher compared to formula fed infants. (B. Wang et al Am. J. Clin Nutr, 2003, 78, 1024-1029). As well as the sialic acid content in saliva of breast fed infant was two times higher than formula fed infants. (H. T. Tram et al Arch. Dis. Child. 1997, 77, 315-318).

Production of Sialylated Oligosaccharides

One of the most abundant and patented group of methods that are developed for the industrial application regarding to sialic acid production are the production of oligosaccharides containing sialic acid. There is a variety of methods for enzymatically producing sialylated oligosaccharides using different trans-sialidases or sialyltransferases and most of them use a dairy source. U.S. Pat. No. 5,374,541 describes a method for producing sialyloligosaccharides. According to this method, β-galactosidase is used to form β-galactosyl glycosides in the presence of CMP-sialic acid and α(2-3)- or α(2-6)-CMP-sialyltransferases to form sialylated oligosaccharides. U.S. Pat. No. 5,409,817 discloses a three enzyme process for producing α(2-3)-sialylgalactosides. According to this process, CMP-sialyltransferases transfer sialic acid from CMP-sialic acid to acceptor molecules, these acceptor molecules become donor molecules for Trypanosoma cruzi α(2-3) trans-sialidase, and CMP-sialic acid is regenerated in the system through the action of CMP-sialic acid synthetase and added free sialic acid. The process described in U.S. Pat. No. 5,409,817 specifically requires the addition of free sialic acid. The free sialic acid is converted to CMP-sialic acid by CMP-sialic acid synthetase, and the sialic acid moiety is transferred to an acceptor molecule by CMP-sialyltransferase. According to the disclosure the formation of these sialylated acceptor molecules is required to drive the α(2-3) trans-sialidase reaction forward. In addition to free sialic acid this method also requires the presence of three enzymes including CMP-sialic acid synthetase and CMP-sialyltransferase. Further, dairy sources and cheese processing waste streams do not contain CMP-sialic acid synthetase.

The more easy method of synthesizing α(2-3)-sialylated conjugates using trans-sialidase is described in CA Pat No 2096923.

U.S. Pat. No. 6,323,008 and U.S. Pat. No. 6,706,497 relate to methods for producing α(2-3) sialyloligosaccharides in a dairy source or cheese processing waste stream by contacting the dairy source or cheese processing waste stream with a catalytic amount of at least one α(2-3) trans-sialidase. In preferred embodiments, the methods of the invention are applied to produce α(2-3)-sialyllactose in a dairy source or cheese processing waste stream. Methods for isolating the α(2-3) sialyloligosaccharides produced according to the methods of the invention are also provided.

U.S. Pat. No. 5,908,766 describes a method of production of saccharides containing sialic acid, wherein β-galactoside-α2,6-sialyltransferase is used for linking sialic acid to the 6-position of a galactose residue in a sugar chain of a glycoconjugate or the 6-position of a galactose residue in a free sugar chain, or to the 6-position of a monosaccharide having a hydroxyl group on carbon at the 6-position and being capable of forming an oligosaccharide or a glycoconjugate.

Another important group of methods for the production of sialic acid containing oligosaccharides includes different sources of sialic acid conjugates as well as other types of enzymatic reactions involved.

For example, typically, α(2-3)-sialyllactose is used as the sialic acid donor in trans-sialidase catalyzed reaction. However, due to limitations such as reversibility and cost, alternative sialic acid donors are needed. S. G Lee et al (Enzyme and Microbial Technology, 2002, 31(6) 742-746) showed that fetuin, a glycoprotein containing abundant sialic acids at the ends of its oligosaccharides, can be used as a sialic acid donor in trans-sialidase catalyzed reaction. Among 166 nmol of total sialic acid in milligrams fetuin, 125 nmol of sialic acid was consumed for the trans-sialidase reaction. The trans-sialidase reaction using fetuin was reversible. The sialyl transfer rate of fetuin to Gal^{β}(1,4)GlcNAc was similar to that of α(2-3)-sialyllactose and approximately 30-40 times greater than that of 4-methylumbelliferryl-α-sialic acid. Trans-Sialidase reaction was performed using 200 mg of fetuin and 34 mg of lactose as a donor and an acceptor, respectively, and 8 mg of product, i.e. α(2-3)-sialyllactose, was purified by gel filtration column. To simplify the purification step, trans-sialidase reaction was carried out by submerging and stirring a dialysis bag containing fetuin and trans-sialidase into a lactose solution.

In addition a number of patents using egg yolk as a source for sialic acids are available.

U.S. Pat. No. 5,233,033 is directed towards a method for producing crude sialic acid, comprising hydrolysis of a delipidated egg yolk and a method for producing high purity sialic acid, which comprises desalting a solution containing sialic acid obtainable by hydrolyzing a delipidated egg yolk, adsorbing sialic acid to an anion exchange resin and then eluting said sialic acid.

In JP Pat. No 08266255, sialic acid-containing oligosaccharide derivatives are obtained from chicken egg yolk upon hydrolysis with a protease. The protease-treatment apparently liberates the oligosaccharide; it is unclear whether all amino acids are removed from the oligosaccharide, or that residual amino acids are still present.

Another patent JP Pat. No 06245784 introduces enzymatic production of compositions containing sialic acid and its derivatives. Those compositions are manufactured by treatment of defatted egg yolk with enzymes (e.g. protease), removal of polymer ingredients by ultra filtration of the water-soluble fractions, and desalting the compounds. Egg yolk powder was stirred with EtOH to give defatted egg yolk. Treatment of 1 ton the defatted egg yolk with Protease A (protease) in H₂O at 50° C. for 8 h, followed by ultrafiltration and desalting gave 300 kg a composition containing free sialic acid 7.5, peptides 25, and sialooligosaccharides 75%.

U.S. Pat. No. 1,523,031 relates to a method for industrial scale extraction and production of lactoserum sialic acid, and said method includes the following steps: hydrolysis step, using lactoserum powder and water to remove protein, regulating pH value, then heating and filtering; superfiltering impurity-removing step, adopting the membrane whose trapping mol. weight is 6000-8000 to make filtration; ion exchange step, using 5-15 L alkaline resin to adsorb the filtrate according to the linear speed of 1-3 m, after water-washing, using pH gradient to make elution; and concentration crystallization step, collecting concentrate, crystallizing, drying or freeze-drying to obtain the invented product.

JP Pat. No 11180993 describes preparation of sialic acid compounds from whey or mother liquor after lactose crystallization. Sialic acid compounds are prepared by passing whey or mother liquor after lactose crystallization through a weakly-basic anion exchange resin column and then eluting the adsorbed sialic acids. Whey or the mother liquor may be passed through a cation exchange resin prior to treatment with the anion exchange resin. Salt strength of the whey or the mother liquor may be previously adjusted at elec. conductivity≦3.0 mS/cm, e.g. by electrodialysis. Cheese whey (solid content 6%) was desalted to elec. conductivity 1.25 mS/cm by an electrodialyzer and passed through IR 120B strongly-acidic cation exchange column and then Diaion WA 10 weakly-basic anion exchange column. The anion exchange column was treated with an aqueous AcONa solution to give an eluate containing 1.3 g/L sialic acids (0.5 g/L in sialyllactose, 0.4 g/L in glycomacropeptides).

Method for comprehensively processing and using poultry egg is presented in CN Pat. No 1511465 and relates to technology of producing with poultry egg various products, such as sialic acid, egg white protein, yolk amino acid, lecithin, lysozyme, etc. The invention features that poultry egg is washed, crushed and separated to obtain egg shell, egg white and yolk; the yolk is produced into sialic acid and lecithin via mixing with water, pH regulation, hydrolysis, ion exchange, spraying to dry, phase separation and other steps; and the egg white is produced into lysozyme and egg white protein via pH regulation, cation exchange and other steps.

Another method for extraction of glycoproteins and sialic acid from whey described in two patents with small modifications U.S. Pat. No. 4,042,576 and U.S. Pat. No. 4,042,575. It includes a development of process for the separation of sialic acid and glycoproteins from dairy or casein factory whey. The proteins are flocculated by thermal treatment, the supernatant is ultrafiltrated and the ultrafiltration retentate is treated by hydrolysis, and the sialic acid is then extracted from the hydrolysis supernatant.

Production of Sialic Acid

In general the sialic acid can be supplied from both enzymatic synthesis and chemical synthesis. Chemical synthesis is not an easy task and it is clearly reflected in prices of commercially available synthetically produced sialic acids. Because the Neu5Ac represents about 95% of total sialic acids in bovine milk and apparently is the most abundant sialic acid in human milk it is of special interest. Also most studies described in literature with sialic acids are done using Neu5Ac.

Biosynthesis of sialic acid proceeds via aldol condensation of N-acetylmannosamione or mannose and pyruvic acid (F. Kimio, Trends in Glycoscience and Glycotechnology, 2004, 16 (89) 143-169, K. Viswanathan, S. Lawrence, S. Hinderlich, K. J. Yarema, Y. C. Lee, M. J. Betenbaugh, Biochemistry, 42 (51), 15215-15225, 2003). It was demonstrated that insect cells can be engineered to produce sialylation substrates and in particular could be used for the production of the sialic acids, e.g Neu5Ac. By varying the specific pathway genes as well as the substrates involved it was determined that particular processing steps can limit the production of sialic acid. Furthermore, a suitable combination of substrate feeding alternatives and expression of various genes can be used to control the levels of sialic acid as well as the type of sialic acid formed. It was reported that Sf9 cells synthesize Neu5Ac and KDN when infected with a baculovirus carrying the gene for sialic acid 9-phosphate synthase in the presence of exogenously fed ManNAc. The levels of Neu5Ac were observed to increase with ManNAc supplementation up to 20 mM fed ManNAc. This increase in Neu5Ac production clearly indicates a limitation in the available ManNAc for Neu5Ac synthesis in Sf9 cells. However, the addition of 50 mM ManNAc gave only a 12% increase in the synthesis of Neu5Ac over the level obtained with 20 mM ManNAc. Thus, a bottleneck in the sialic acid pathway is present in insect cells such that increasing the level of ManNAc present in the medium above 20 mM does not cause a significant enhancement in the amount of Neu5Ac generated. This bottleneck could exist either at the step involving ManNAc transport into the cells or in the metabolic conversion of ManNAc to substrates which can be utilized by the sialic acid synthesizing enzyme. Nonetheless, the intracellular Neu5Ac content was still over 100 times higher in the AcSAS-infected lysates as compared to control culture lysates. However, the presence of detectable Neu5Ac in control cultures suggests that insect cells may contain very low endogenous levels of the enzymes for sialic acid synthesis. The gene for sialic acid synthesis indeed has been detected in Drosophila melanogaster although the endogenous enzymatic activity was undetectable in Schneider S2 cell lines. KDN, an alternate sialic acid, was also generated following AcSAS infection. The ratio of KDN to Neu5Ac decreased drastically following ManNAc feeding due to a rapid increase in the synthesis of Neu5Ac, indicating that ManNAc-6-P is the preferred substrate of SAS. In spite of clear indication of NeuAc production by insects cells this method, to our knowledge, is not used commercially. This might be caused by a too high process cost.

Some of the methods for production the sialic acid containing compounds do not use enzymatic reactions and rather chemical methods are applied. Some of those methods are described in patent literature. U.S. Pat. No. 5,270,462 relates to a process for manufacturing a composition containing sialic acids. The process comprises the steps of: (a) adjusting cheese whey or rennet whey to a pH of 2-5; (b) contacting the whey with a cation exchanger, to produce an exchanger-passed solution; (c) adjusting the pH of the exchanger-passed solution to a pH of 4 or lower; and then (d) concentrating and/or desalting the exchanger-passed solution. The possibility to produce a composition having high sialic acids was claimed.

Sialidases

Sialidases (neuraminidases, EC 3.2.1.18) hydrolyze the terminal, non-reducing, sialic acid linkage in glycoproteins, glycolipids, gangliosides, polysaccharides and synthetic molecules. Some sialidases, called transsialidases, are also capable to perform transfer-reactions in which they transfer the sialic acid residue from one molecule to another. Sialidases are common in animals of the deuterostomate lineage (Echinodermata through Mammalia) and also in diverse microorganisms that mostly exist as animal commensals or pathogens. Sialidases, and their sialyl substrates, appear to be absent from plants and most other metazoans. Even among bacteria, sialidase is found irregularly so that related species or even strains of one species differ in this property. Sialidases have also been found in viruses and protozoa (Traving et al Cell Mol Life Sci (1998) 54, 1330-1349) and sialidase activity has also been found in fungi (Uchida et al 1974, Biochim Biophys Acta 350, 425-431). Micro-organisms containing sialidases often live in contact with higher animals as hosts, for example as parasites. Here they may have a nutritional function enabling their owners to scavenge host sialic acids to use as a carbon source. For some microbial pathogens, sialidases are believed to act as virulence factors. Yet, the role of sialidases as factors in pathogenesis is controversial. On the one hand they confirm the impact of pathogenic microbial species like Clostridium perfringens. On the other hand, these enzymes are factors common in the carbohydrate catabolism of many non-pathogenic species, including higher animals. They do not, however, exert a direct toxic effect (Traving et al Cell Mol Life Sci (1998) 54, 1330-1349). Instead, their detrimental effect depends on the massive amount of enzyme that is released into the host together with other toxic factors upon induction by host sialic acids under non-physiological conditions.

The mammalian sialidases are normally approximately 40-45 kDa in size. Attempts to over-express and produce mammalian sialidases to industrially interesting amounts have not been reported. Human sialidases can be lysosomal, cytosolic or membrane bound enzymes (Achyuthan and Achyuthan (2001) Comp. Biochem. Phys. Part B, 129, 29-64). The lysosomal sialidases are glycosylated enzymes. Sialidases contain conserved motifs. The most prominent conserved motif is the so called Asp-box, which is a stretch of amino acids of the general formula —S—X-D-X-G-X-T-W— where X represents a variable residue. This motif is found four to five times throughout all microbial sequences with the exception of viral sialidases, where it is found only once or twice or is even absent. The third Asp-box is more strongly conserved than are Asp-boxes 2 and 4. The space between two sequential Asp-boxes is also conserved between different primary structures (Traving et al Cell Mol Life Sci (1998) 54, 1330-1349). The Asp-boxes probably have a structural role and are probably not involved in catalysis. In contrast to the Asp-boxes, the FRIP-motif is located in the N-terminal part of the amino acid sequences. It encompasses the amino acids —X—R—X—P— with the arginine and proline residues absolutely conserved. The arginine is directly involved in catalysis by binding of the substrate molecule. Also important for catalytic action is a glutamic acid rich region between asp-boxes 3 and 4 as well as two further arginine residues (Traving et al Cell Mol Life Sci (1998) 54, 1330-1349)

Microbial sialidases can be classified into two groups according to their size: small proteins of around 42 kDa and large ones of 60-70 kDa. The primary structure of the large sialidases contains extra stretches of amino acids between the N-terminus and the second Asp-box as well as between the fifth Asp-box and the C-terminus. It is believed that they contribute to the broader substrate specificity of the large sialidases. Like the mammalian sialidases, the bacterial counterparts contain the F/YRIP motif and several Asp-boxes. Bacterial sialidases are often implicated in mucosal infections and virulance. Because of this, the larger bacterial sialidases are not regarded suitable for the use as processing aid in food or pharma applications. Small sialidases (same size as the mammalian sialidases) have been identified in bacteria, as indicated above. I.e. Clostridium perfringens contains a small sialidase with a size of ˜40 kDa, without the extensions common to sialidases in other bacteria. This Clostridium sialidase is however not secreted by the bacterium, and is therefore also not involved in virulance (Roggentin et al. (1995) Biol Chem Hoppe Seyler 376, 569-575). It is tempting to speculate that only the bacterial sialidases with extra extensions are involved in pathogenicity. Overexpression of bacterial sialidases in E. coli generally leads to low productivity; the small Clostridium sialidase could only be produced to 1 mg/l as intracellular protein in E. coli (Kruse et al. (1996) Protein Expr Purif. 7, 415-422). There is therefore a clear need for a well-produced small, non-virulent sialidase for applications in food and pharma.

### SUMMARY OF THE INVENTION

The present invention relates to a composition which comprises:
an oligosaccharide,
sialyloligosaccharide in an amount of 0 to 1 wt %, preferably less than 0.1 wt %, of the total amount of oligosaccharide present,
free sialic acid.

Preferably this composition comprises sialyloligosaccharide in an amount of less than 1 wt %, preferably less than 0.1 wt %, of the total amount of oligosaccharide present and most preferably is substantially free of sialyloligosaccharide.

The composition of the invention preferably comprises free sialic acid in an amount of more than 0.001 wt %, preferably more than 0.01 wt %, still more preferably more than 0.1 wt %, and most preferably more than 1 wt %, of the total amount of oligosaccharide and free sialic acid present.

The composition of the invention preferably comprises less than 0.5 wt % (dry matter) of fucose, more preferably comprises less than 0.1 wt % (dry matter) of fucose, and most preferably comprises less than 0.01 wt % (dry matter) of fucose,

The composition is advantageously a prebiotic composition, suitable for human consumption.

The composition of the invention can be produced in a process which comprises
subjecting a first suitable substrate to a suitable enzyme to produce an oligosaccharide, and
subjecting a second suitable substrate to a sialidase to produce free sialic acid.
   The process of the invention can be done in several ways for example the first and second substrate can be identical, and than both steps can take place in one reactor. In another embodiment the steps will take place after each other. In still another embodiment the steps take place separately and the sialic acid and oligosaccharide are combined.
   According to another aspect of the invention immobilized sialidase is disclosed and a process to produce sialic acid whereby the sialidase used is immobilized. Also the present invention relates to food, including a drink, or feed which comprises the composition of the invention, or a composition produced with the process of the invention.

This invention relates to an enzymatic method using a novel sialidase to prepare a prebiotic composition containing prebiotic oligosaccharides and free sialic acid. The prebiotic composition is characterized by the following composition:
It is free of sialyloligosaccharides (<1.0 wt %, preferably <0.1 wt % of total oligosaccharides in the preaparation)
The amount of free sialic acid is preferably >0.001% of the combined weight of sialic acid and oligomeric prebiotics in the prebiotic composition, more preferably 0.01% of the combined weight of sialic acid and oligomeric prebiotics in the prebiotic composition, even more preferably 0.1% of the combined weight of sialic acid and oligomeric prebiotics in the prebiotic composition and most preferably >1% of the combined weight of sialic acid and oligomeric prebiotics in the prebiotic composition

The method consists of contacting a solution containing a substrate from which prebiotic oligosaccharides can be formed in combination with a substrate from which sialic acid can be released.

The substrate for the prebiotic oligosaccharides can be one or a combination of the following substrates: a dairy composition, lactose, sucrose, inulin, maltose, soybean, starch, glucose syrup, or xylan, preferably a dairy composition. The production of oligosaccharides is known in the art and for example processes described in the background of the invention can be used.

The substrate for the sialic acid is can be one or a combination of the following substrates: a dairy composition, egg yolk or defatted egg yolk, preferably a dairy composition.

The release of sialic acid is performed using the sialidase enzyme, preferably the enzyme described in this application. The formation of prebiotic oligosaccharides may be performed with any enzyme, useful for the chosen substrate.

### DETAILED DESCRIPTION OF THE INVENTION

The prebiotic composition of the invention is industrially attractive because it combines the beneficial effects of prebiotic oligosaccharides with those of free sialic acid. The advantage over currently available and described sialyloligosaccharides and their preparation using transsialidases is that in the current invention the ratio of free sialic acid to the prebiotic oligosaccharide can be chosen as preferred. In addition, sialic acid can be combined with any type of preferred prebiotic oligosaccharides, whereas in the preparation of sialyloligosaccharides, only those oligosaccharides can be used that can function as substrate for the transsialidases.

The present invention is based on our insight that a prebiotic composition comprises sialic acid as well as oligosaccharides. In the prior art thereto the sialic acid was built into the oligosaccharides. This resulted in the production of sialyloligosaccharides, which comprises both elements. Although these sialyloligosaccharides are very useful products, the production thereof is apart from being complicated also very expensive, and at the moment no economical attractive route is known. According to the present invention a cheap alternative is offered which has all the positive effects of sialyloligosaccharides and can be produced in a simple and economically attractive way. In all cases, oligosaccharide preparations are described in the prior art are described as such or as a combination of free sialic acid and sialic acid containing oligosaccharides. In some cases where transsialidases are used, the methods seem to be optimized to reduce levels of free sialic acid as much as possible in favour of the uptake of sialic acid in the sialyloligosaccharides. The present invention is based on the insight that the combination of oligosaccharide, free of sialyloligosaccharide, and free sialic acid has the same benefits as sialyloligosaccharides for humans or other mammalians.

No sialidases have been identified at the molecular level (that is, no amino acid sequence or gene sequence has been described) in plants and fungi until now, although sialidase activity has been demonstrated in fungi (Uchida et al 1974, Biochim Biophys Act 350, 425-431).

Especially the finding of a secreted fungal sialidase is found to be beneficial, since secreted enzymes can be easily over-expressed and purified in large quantities from a fungal culture. This reduces the cost-price for production of a sialidase dramatically. In addition, it would allow the cost-effective production of sialic acid from e.g. dairy compositions and egg yolk. This would open the way for the production of a new generation of prebiotic compositions, containing a combination of non-sialylated prebiotic oligosaccharides and free sialic acid. The combination of free sialic acid and non-sialylated prebiotic oligosaccharides has to our knowledge not been described.

Novel Sialidase

The present invention relates to a method to produce a prebiotic composition containing free sialic acid and prebiotic oligosaccharides such as but not limited to galacto-oligosaccharides, fructo-oligosaccharides and lactulose. The enzymatic, cost effective production of sialic acid requires the availability of a well produced sialidase. Sialidase is commercially only available in small quantities at high price. Sigma company provides sialidases at prices of [symbol] 15.20 to approximately [symbol] 1500 for mg-quantities of the enzyme, which does not allow for the cost-effective production of sialic acid from natural sources. In this application we describe the identification of a new fungal sialidase which has been identified in the fungus Penicillium chrysogenum.

Advantageously the present invention meets the demand for a sialidase that can be produced in high amounts. Preferably, such a sialidase is secreted from the host cell. Active secretion is of paramount importance for an economical production process because it enables the recovery of the enzyme in an almost pure form without going through cumbersome purification processes. Overexpression of such an actively secreted sialidase by a food grade fungal host such as Aspergillus, yields a food grade enzyme and a cost effective production process, and is therefore preferable. The presently secreted sialidase is for the first time found in filamentous fungi. Processes are disclosed for the production of sialidase in large amounts by the food-grade production host Aspergillus niger.

From an economic point of view there exists a clear need for an improved means of producing sialidases in high quantities and in a relatively pure form, compared to the poor productivity of the mammalian and bacterial sialidases. A preferred way of doing this is via the overproduction of such a sialidase using recombinant DNA techniques. A particularly preferred way of doing this is via the overproduction of a fungal derived sialidase and a most preferred way of doing this is via the overproduction of an Penicillium derived sialidase. To enable the latter production route unique sequence information of an Penicillium derived sialidase is essential. More preferable the whole nucleotide sequence of the encoding gene has to be available. We have identified such sialidase enzyme in the genome of Penicillium chrysogenum. Its amino acid sequence is given as SEQ ID No 3, its corresponding genomic nucleotide sequence in SEQ ID no 1 and its coding sequence in SEQ ID no 2. The novel enzyme is well produced in Aspergillus niger and has sialidase activity.

Dairy Composition

A dairy composition according to the invention may be any composition comprising cows milk constituents. Milk constituents may be any constituent of milk (other than water) such as milk fat, milk protein, casein, whey protein and lactose. A milk fraction may be any fraction of milk such as e.g. skim milk, butter milk, whey, cream, milk powder, whole milk powder, skim milk powder. In a preferred embodiment of the invention the dairy composition comprises milk, skim milk, butter milk, whole milk, whey, cream, or any combination thereof. In a more preferred embodiment the dairy composition consists of milk, such as skim milk, whole milk, cream or any combination thereof.

In further embodiments of the invention, the dairy composition is prepared, totally or in part, from dried milk fractions, such as e.g. whole milk powder, skim milk powder, casein, caseinate, total milk protein or buttermilk powder, or any combination thereof. The dairy composition also includes whey solutions as they are generated during cheese manufacture. Any cheese manufacture process will generate a whey solution, and the composition varies with the cheese manufacturing protocol.

In yet another embodiment of the invention, the dairy composition is prepared, totally or in part, from milk or milk fractions that have been subjected to proteolytic degradation to prepare milk protein hydrolysates. These milk protein hydrolysates may be combined with milk or milk fractions to form a dairy composition.

According to the invention the dairy composition comprises cow's milk and or one or more cow's milk fractions. The cow's milk fractions may be from any breed of cow (Bos Taurus (Bos taurus taurus), Bos indicus (Bos indicus taurus) and crossbreeds of these. In one embodiment the dairy composition comprises cow's milk and/or cow's milk fractions originating from two or more breeds of cows. The dairy composition also comprises milk from other mammals that are used for cheese preparation, such as milk derived from goat, buffalo or camel.

The dairy composition for production of cheese may be standardized to the desired composition by removal of all or a portion of any of the raw milk components and/or by adding thereto additional amounts of such components. This may be done e.g. by separation of milk into cream and milk upon arrival to the dairy. Thus, the dairy composition may be prepared as done conventionally by fractionating milk and recombining the fractions so as to obtain the desired final composition of the dairy composition. The separation may be made in continuous centrifuges leading to a skim milk fraction with very low fat content (i.e. <0.5%) and cream with e.g. >35% fat. The dairy composition may be prepared by mixing cream and skim milk. In another embodiment the protein and/or casein content may be standardized by the use of ultra filtration. The dairy composition may have any total fat content that is found suitable for the cheese to be produced by the process of the invention.

In one embodiment of the invention, calcium is added to the dairy composition. Calcium may be added to the dairy composition at any appropriate step before and/or during cheese making, such as before, simultaneously with, or after addition of starter culture. In a preferred embodiment calcium is added both before and after the heat treatment. Calcium may be added in any suitable form. In a preferred embodiment calcium is added as calcium salt, e.g. as CaCl₂. Any suitable amount of calcium may be added to the dairy composition. The concentration of the added calcium will usually be in the range 0.1-5.0 mM, such as between 1 and 3 mM. If CaCl₂ is added to the dairy composition the amount will usually be in the range 1-50 g per 100 liter of dairy composition, such as in the range 5-30 g per 1000 liter dairy composition, preferably in the range 10-20 g per 100 liter dairy composition.

Probiotics

The composition of the invention preferably also comprises a probiotic.

Probiotics or probiotic compositions are defined as live microbial food ingredients that when administered in adequate amounts confer a health benefit on a host. The criteria for a probiotic or a probiotic composition are: survival through the gastrointestinal tract, non-toxic, non-pathogenic, accurate taxonomic identification, ability to proliferate and be metabolically active in the gastrointestinal tract, demonstrable health benefit, such as immune modulation, improvement of the balance of bacteria in the gastrointestinal tract, stability of strain during processing, storage and delivery, production and viability at high cell densities.

Immobilized Enzymes

An immobilised enzyme is an enzyme which is attached to an inert, insoluble material.

In the processing of foods or food ingredients, enzymes have distinct advantages over chemical catalysts of which most notable are substrate specificity and activity under mild conditions of temperature and pH. However, the cost of using soluble enzymes is a drawback. For that reason, there is interest in the use of immobilized enzymes. These immobilized enzymes are physically confined or localized in a certain defined region of space with retention of their catalytic activities, and they can be used repeatedly and continuously. Advantages of enzyme immobilization include:
reuse or continuous use of the catalyst, thereby reducing both capital and recurrent process costs
absence of the enzyme from the product, thus potentially allowing for a wider range of enzymes than those normally permitted in foods
ease of terminating the reaction without drastic measures such as heat denaturation or extreme pH
in some cases, greater thermal and pH stability, prevention of self-digestion by proteases, and stabilization of its tertiary structure, potentially prolonging its useful life
less product inhibition, and more substrate depletion with continuous processes, giving faster conversion

The main disadvantages are the cost of producing the immobilized enzyme, including the cost of the support, and altered reaction kinetics, which often result from diffusional restrictions, pH shifts, and partitioning. Furthermore, a perfect, universal immobilization method does not exist; each end use requires evaluation of the individual steps according to criteria such as the purpose of immobilization, activity, stability, simplicity, and economic feasibility.

Many different methods for enzyme immobilization exist, with a main classification in methods for insoluble enzymes and methods for soluble enzymes. Further classification of the methods for insoluble enzymes is binding or entrapment of the enzyme. For each of these methods, different techniques exist. For binding of the enzyme to a carrier the following techniques are known:
physical adsorption: the enzyme adheres to the surface of a support by means of physical interactions such as Van der Waals forces, hydrogen bonding, or hydrophilic-hydrophobic effects
ionic binding: the binding forces are ion-ion interactions, which are stronger than in simple physical adsorption
chelate binding: the chelating properties of a transition metal such as titanium or zirconium are employed to couple enzymes to an organic material or an inorganic support
biospecific binding: biospecific interactions between enzymes and other molecular species (e.g. lectins, or antibodies, are used for binding the enzyme
covalent binding: a water-insoluble carrier can be covalently bound to the enzyme via the reactive side groups of amino acid residues (e.g. amino, hydroxyl, thiol, or phenolic groups) that are not associated with the active site or the substrate binding site

Other techniques of binding the enzyme are cross-linking and enzyme copolymerization. In cross-linking the enzyme is immobilized by cross-linking it to other enzyme molecules or to an inert protein such as albumin, and precipitate the resulting aggregate. This method can also be used in combination with a carrier such as a membrane, where the physically adsorbed enzymes are cross-linked on the membrane surface. In enzyme copolymerization the enzyme is copolymerized with a polymer matrix, e.g. enzymes are vinylated with acylating or alkylating monomers and copolymerized with other monomers.

Entrapment of an enzyme may be reagrded as the physical confinement of an enzyme in a semipermeable matrix, which must be tight enough for the enzyme to be retained but must allow permeation of substrate and product(s). Entrapment techniques are:
gel entrapment: free enzyme is entrapped within the interstatial spaces of a cross-linked, water-insoluble polymeric gel (e.g. alginate, agar, K-carrageenan)
microencapsulation: enzymes are immobilized by enclosing them in membranes that are permeable to the substrate and the product(s), usually an emulsion of an organic phase and an aqueous enzyme containing phase in the presence of a surfactant is prepared, to which a membrane forming polymer is then added, the resulting microcapsules generally have a diameter of 1 to 100 μm
reverse micelles: amphiphilic surfactant molecules can form reverse micelles in hydrocarbon solvents, the enzymes or contained in the water pools of the micelles, and they retain their biological activity resulting from protection from the organic solvents by the surfactant envelope.

Methods for immobilization of soluble enzymes have the advantage that the enzyme is in its native state and microenvironment, which does not result in decrease of enzyme activity. This can be achieved by separating the enzyme solution from the substrate and product by a semipermeable membrane, which allows substrate and product diffusion and physically confines the larger enzyme molecule. This can be achieved by flat sheet ultrafiltration or microfiltration membranes or hollow-fiber membranes. In this case co-factors able to diffuse through the membrane can be retained in the reaction zone by coupling them to larger molecules. A final method of immobilization is the use of variable solubility of an immobilized enzyme under different conditions, known as soluble-insoluble immobilized enzymes. An example is a cellulase immobilzed on poly(L-glutamic acid), which is soluble in neutral and alkaline solution, but can be precipitated by lowering the pH without loss of enzyme activity (Clemmings et al, 1999, Wiley Encyclopedia of Food Science and Technology (2^{nd} edition) Volumes 1-4, John Wiley & Sons, p 1342-1345; Prenosil et al, 2007, Ullmann's Encyclopedia of Industrial Chemistry (7^{th} edition)).

Preparation of Prebiotic Compositions Containing Free Sialic Acid.

Methods for the enzyme catalyzed preparation of prebiotic oligosaccharides have been described previously in this application. The preparation of prebiotic compositions containing free sialic acid can be prepared using the same technologies, starting from the same starting materials as described previously but supplemented with one or more sialic acid containing substrates. Such substrates have been described previously in this application and include dairy compositions and egg protein preparations. The prebiotic composition containing free sialic acid is preferably prepared in a one step process in which the substrates for the preparation of the prebiotic oligosacchrides and the sialic acid are mixed and treated with a combination of enzymes, in which one of the enzymes is a sialidase. In a preferred embodiment, the prebiotic composition containing free sialic acid is prepared from a dairy composition using a β-galactosidase and a sialidase. The enzymes may be added to the substrates, resulting in a homogeneous solution containing both enzymes and substrates. After proper reaction time when free sialic acid an oligosaccharides have been formed, the enzymes can be inactivated using e.g. heat treatment or other methods known to the person skilled in the art to inactivate enzymes. The prebiotic preparation containing free sialic acid may be further processed to concentrate the reaction mixture or to remove undesirable components. Suitable techniques are known to the person skilled in the art and include but are not limited to ultra filtration, spray drying and chromatographic techniques.

In yet another embodiment, the enzymatic formation of sialic acid and enzymatic oligosaccharide may be subsequent reactions. The formation of sialic acid may be performed prior to the formaton fo oligosaccharides, alternatively the sialic acid may only be liberated after forming of the oligosaccharides.

Immobilized enzymes may also be used for the enzymatic preparation of the prebiotic composition containing free sialic acid. The immobilized enzymes can be suspended in the reaction mixture to achieve the desired formation of the prebiotic composition. The enzymes are than easily removed by filtration after which they can be re-used. Alternatively, the immobilized enzymes can be packed in a column, and the substrate solution is than pumped over the column. Residence time of the substrates in the column can be tuned to obtain the desired formation of the prebiotic composition containing free sialic acid. Such process have been described, e.g. for the production of galactooligosachharides (see e.g. Ekhart et al, J Food Protection 53, 262-268). Also in the case of immobilized enzymes, the enzymatic treatments may be separated in time, as described before.

In a preferred embodiment, the substrate is a mixture containing the relevant precursors of both the prebiotic oligosaccharides and the sialic acid. In another embodiment, the substrates for oligosaccharide formation and sialic acid formation may be present in separate containers or vials. This allows the enzymatic generation of oligosaccharides and sialic acid separately, followed by mixing the two reaction products leading to a composition containing a prebiotic oligosaccharide with free sialic acid.

### LEGENDS TO THE FIGURE

FIG. 1: ZJW expression vector named pGBFINZJW

FIG. 2: Release of sialic acid from whey (squares) and milk (circles) substrates after incubation them with sialidase enzyme at 0.04 u/ml. The dashed lines are corresponding background measurements where instead of sialidase the milliQ water was added.

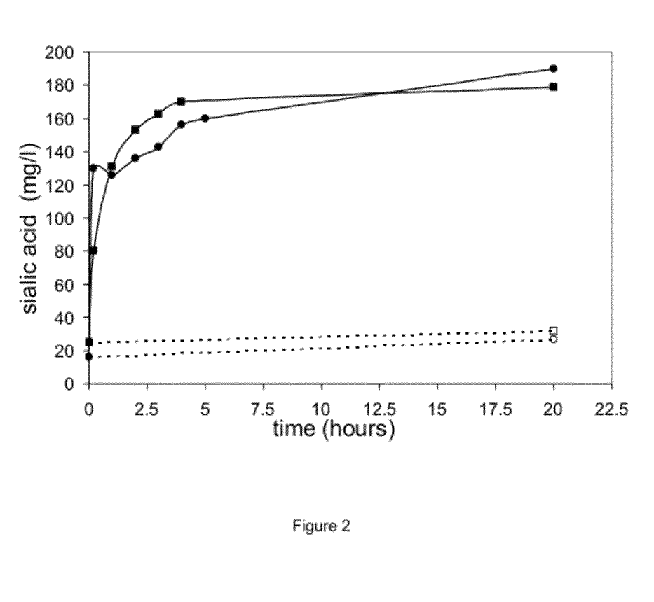

### EXAMPLES

### Example 1

### Cloning and Expression of the Sialidase Gene ZJW

Penicillium chrysogenum strain CBS 455.95 was grown for 3 days at 30 degrees Celsius in PDB (Potato dextrose broth, Difco) and chromosomal DNA was isolated from the mycelium using the Q-Biogene kit (catalog nr. 6540-600; Omnilabo International BV, Breda, the Netherlands), using the instructions of the supplier. This chromosomal DNA was used for the amplification of the coding sequence of the sialidase gene using PCR.

To specifically amplify the sialidase gene ZJW from the chromosomal DNA of Penicillium chrysogenum strain CBS 455.95, two PCR primers were designed. Primer sequences were partly obtained from a sequence that was found in the genomic DNA of Penicillium chrysogenum CBS 455.95 and is depicted in SEQ ID NO: 1. We found that this sequence has homology with sialidase sequences of Actinomyces and Arthrobacter. However, no homologous fungal sialidases have been described yet. It is therefore surprising that we were able to find a gene encoding a secreted sialidase from a fungus. We describe here for the first time the efficient expression and characterization of a secreted fungal sialidase. The protein sequence of the complete sialidase protein, including potential pre- and pro-sequences is depicted in SEQ ID NO: 3. The advantage of the fungal enzyme compared to the bacterial homologues is that the fungal enzyme can be easily overexpressed and secreted in amounts that are relevant for applications in the food industry.

Zjw-dir
5′-CCCTTAATTAACTCATAGGCATCATGCTATCTTCATTGATGTATTT
Zjw-rev
5′-TTAGGCGCGCCGTACATACATGTACACATAGACC

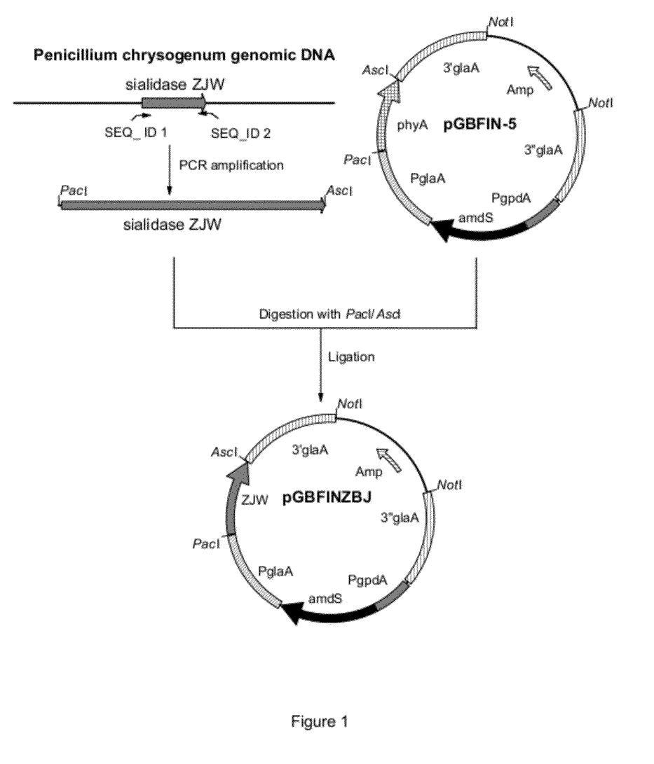

The first, direct PCR primer (ZJW-dir) contains 23 nucleotides ZJW coding sequence starting at the ATG start codon, preceeded by a 23 nucleotides sequence including a Pacl restriction site (SEQ ID NO:4). The second, reverse primer (ZJW-rev) contains nucleotides complementary to the reverse strand of the region downstream of the ZJW coding sequence preceeded by an Ascl restriction site (SEQ ID NO:5). Using these primers we were able to amplify a 1.4 kb sized fragment with chromosomal DNA from Penicillium chrysogenum strain CBS 455.95 as template. The thus obtained 1.4 kb sized fragment was isolated, digested with Pacl and Ascl and purified. The Pacl/Ascl fragment comprising the ZJW coding sequences was exchanged with the Pacl/Ascl phyA fragment from pGBFIN-5 (WO 99/32617). Resulting plasmid is the ZJW expression vector named pGBFINZJW (see FIG. 1). The expression vector pGBFINZJW was linearized by digestion with Notl, which removes all E. coli derived sequences from the expression vector. The digested DNA was purified using phenol: chloroform:isoamylalcohol (24:23:1) extraction and precipitation with ethanol. These vectors were used to transform Aspergillus niger CBS513.88. An Aspergillus niger transformation procedure is extensively described in WO 98/46772. It is also described how to select for transformants on agar plates containing acetamide, and to select targeted multicopy integrants. Preferably, A. niger transformants containing multiple copies of the expression cassette are selected for further generation of sample material. For the pGBFINZJW expression vector 30 A. niger transformants were purified; first by plating individual transformants on selective medium plates followed by plating a single colony on PDA (potato dextrose agar: PDB+1.5% agar) plates. Spores of individual transformants were collected after growth for 1 week at 30 degrees Celsius. Spores were stored refrigerated and were used for the inoculation of liquid media.

An A. niger strain containing multiple copies of the expression cassette was used for generation of sample material by cultivation of the strain in shake flask cultures. A useful method for cultivation of A. niger strains and separation of the mycelium from the culture broth is described in WO 98/46772. Cultivation medium was in CSM-MES (150 g maltose, 60 g Soytone (Difco), 15 g (NH₄)₂SO₄, 1 g NaH₂PO₄.H₂O, 1 g MgSO₄.7H₂O, 1 g L-arginine, 80 mg Tween-80, 20 g MES pH6.2 per liter medium). 5 ml samples were taken on day 4-8 of the fermentation, centrifuged for 10 min at 5000 rpm in a Hereaus labofuge RF and supernatants were stored at −20° C. until further analyses.

It became clear that transformants containing the pGBFINZJW vector secreted a protein of apparent molecular weight of approximately 50 kDa when analyzed with SDS-PAGE. Since this is slightly larger than the molecular weight that is predicted from the protein sequence, we presume that after removal of the signal sequence some glycosylation takes place when Penicillium chrysogenum sialidase ZJW is secreted from Aspergillus niger.

Selected strains can be used for isolation and purification of a larger amount of fungal sialidase, when fermentation and down-stream processing is scaled up. This enzyme can than be used for further analysis, and for the use in diverse industrial applications.

### Example 2

### Purification and Characterization of the Sialidase ZJW

Sialidase was produced via fermentation as described in Example 1. Enzyme activity was measured using the Amplex Red neuraminidase assay kit (obtained from Invitrogen). Culture filtrate (100 ml) was diluted with milliQ-water to a conductivity of 4.8 mS/cm and concentrated to 70 ml by ultrafiltration using a Biomax-10 membrane (obtained from Millipore). The pH was adjusted to 6.0 using NaOH and the sample was loaded on a 5 ml HiTrapQ ion exchange column (obtained from Amersham, 5 ml/min), equilibrated in 20 mM sodium citrate (pH6.0). The flow through of the column, containing the sialidase, was collected and dialyzed against 25 mM Tris, HCl (pH7.0) and loaded on a 5 ml HiTrap Q FF (5 ml/min), equilibrated in the same buffer. The sialidase was present in the flow-through fraction and was collected. The enzyme solution was then dialyzed against 30 mM sodium citrate (pH4.0, buffer A) and applied on a 5 ml HiTrap SP column (obtained from Amersham, 5 ml/min), equilibrated in buffer A. After loading the enzyme, the column was washed with 3 column volumes of buffer A and the enzyme was eluted with a linear gradient of 20 column volumes from buffer A to buffer B (buffer B: 30 mM sodium-citrate, pH4.0 containing 1 M NaCl). Sialidase-containing fractions were identified and pooled. Protein concentration was determined with the Bradford reagent (obtained from Sigma), using bovine serum albumin as reference protein. The protein was >95% pure as judged by the absence of contaminating bands on sodium-dodecyl polyacrylamide gel electrophoresis. The sialidase migrates at an apparent molecular weight of 47 kD, which is slightly higher than the molecular weight of 42.7 kD, calculated on basis of the predicted amino acid sequence. The enzyme preparation is does not show proteolytic activity on a series of substrates ZAAXpNA (Z=benzoyl group, A=alanine, X=any amino acid residue, pNA=para-nitroanilide), indicating absence of endo-protease activity.

### Example 3

### Liberation of Free Sialic Acid from Milk and Whey

Free sialic acid can be analyzed by means of reverse-phase HPLC, using fluorescence detection with excitation at 310 nm and emission 448 nm after labelling with DMB compound. This method was recently described in literature (M. J. Martin et al Anal. Bional. Chem., 2007, 387, 2943-29-49) and allowed fast and accurate determination of free sialic contents in samples.

### Sample Preparation

Milk was reconstituted by using NILAC low heat skim milk powder (NIZO, The Netherlands); whey was obtained from a local cheddar making facility. The milk and whey samples were treated as follows: milk and whey was incubated separately with sialidase ZJW (0.4 U/ml) at room temperature (20-21° C.) and the reaction was terminated at different moments of time by heating the samples in water bath at 95° C. for 5 minutes. A series of several sialidase ZJW concentrations and incubation times were performed. The samples used for HPLC analysis need to be free from proteins. Therefore, samples were filtered with a Nanosep ultra filtration Eppendorff (10 KD) using centrifugation for 15 minutes at 14000 g. After centrifugation, the free protein supernatant was collected and diluted 100 times with milliQ water in order to measure the sialic acid in a proper range of concentrations (measuring range using RP HPLC method is 5 fmol-5 nmol of sialic acid).

### Labelling Procedure

The labelling was carried out by transferring of 25 μL of the diluted supernatant into Eppendorf tubes and mixing well with 100 μL of reaction mixture (DMB: Coupling solution: milliQ=1:5:4). The samples were incubated under protection from light in a thermomixer at 50° C. for 2.5 hours with continuous mixing at 500 rpm. The reaction was stopped by cooling on ice. From the reaction mixture, 10 μL was injected to the HPLC system for the analysis.

### HPLC System

Injector: Waters 2690 separation module. Detector: Waters 474 Scanning Fluorocence detector (Ex: 310 nm. Em: 448 nm). Column: PALPAK Type R from TAKARA BIO INC with dimension (L×ID) 250×4.6 mm flow rate: 0.9 ml/min. Run time: 30 min. Solvent: Acetonitrile/methanol/milliQ=9/7/84 (v/v/v) Injection volume: 10 μL. Column temperature: 40° C.

The free sialic acid levels increased in time, reaching a concentration of approximately 220 mg/L after 4 hours indicating; after 20 hours of incubation, levels of free sialic acid reached 250 mg/ml indicating that essentially all sialic acid has been released (for ref: Takeuchi et al, 1985, Agric Biol Chem 49, 2269-2276). Apparently, sialidase ZJW is able to effectively and quickly liberate all available sialic acid from κ-casein in milk and from the GMP-protein in whey.

1. A composition which comprises: an oligosaccharide, sialyloligosaccharide in an amount of 0 to 1 wt %, and free sialic acid.

2. A composition of claim 1 which comprises sialyloligosaccharide in an amount of less than 0.1 wt % of the total amount of oligosaccharide present.

3. A composition according to claim 1 which comprises free sialic acid in an amount of more than 0.001 wt %, preferably more than 0.01 wt %, still more preferably more than 0.1 wt %, and most preferably more than 1 wt %, of the total amount of ligosaccharide and free sialic acid present.

4. The composition according to claim 1 which comprises less than 0.5 wt % (dry matter) of fucose, more preferably comprises less than 0.1 wt % (dry matter) of fucose, and most preferably comprises less than 0.01 wt % (dry matter) of fucose,

5. A composition according to claim 1 which is a prebiotic composition.

6. A process to produce a composition according to claim 1 which comprises subjecting a first suitable substrate to a suitable enzyme to produce an oligosaccharide, and subjecting a second suitable substrate to a sialidase to produce free sialic acid.

7. A process according to claim 6 wherein the first and second substrate are identical.

8. A process according to claim 6 wherein subjecting a first suitable substrate to a suitable enzyme to produce an oligosaccharide, and subjecting a second suitable substrate to a sialidase to produce free sialic acid takes place in one reactor.

9. A process according to claim 6 wherein subjecting a first suitable substrate to a suitable enzyme to produce an oligosaccharide, and subjecting a second suitable substrate to a sialidase to produce free sialic acid is taken place separately, whereafter the oligosaccharide and free sialic acid are combined.

10. A process according to claim 6 wherein subjecting a first suitable substrate to a suitable enzyme to produce an oligosaccharide, and subjecting a second suitable substrate to a sialidase to produce free sialic acid takes place subsequently in one reactor.

11. Process to produce sialic acid wherein the sialidase is immobilized.

12. Food, including a drink, or feed which comprises the composition of claim 1, or a composition produced by a process comprising subjecting a first suitable substrate to a suitable enzyme to produce an oligosaccharide, and subjecting a second suitable substrate to a sialidase to produce free sialic acid.

13. A composition which comprises the composition of claim 1 or the composition produced by a process comprising subjecting a first suitable substrate to a suitable enzyme to produce an oligosaccharide, and subjecting a second suitable substrate to a sialidase to produce free sialic acid; and a probiotic.
